(19) United States
(12) Patent Application Publication
(10) Pub. No.: US 2013/0010262 A1
(43) Pub. Date: Jan. 10, 2013

(54) IMAGING APPARATUS AND IMAGING METHOD

(75) Inventors: Makoto Sato, Tokyo (JP); Yukio Sakagawa, Tokyo (JP)

(73) Assignee: CANON KABUSHIKI KAISHA, Tokyo (JP)

(21) Appl. No.: 13/634,623

(22) PCT Filed: Mar. 29, 2011
(86) PCT No.: PCT/JP2011/001875

§ 371 Date: Sep. 13, 2012

(30) Foreign Application Priority Data
Mar. 31, 2010 (JP) 2010-082814

(51) Int. Cl. A61B 3/14; G01B 9/02

(52) U.S. Cl. 351/206; 356/479

(57) ABSTRACT

An imaging apparatus capturing optical coherence tomographic images of a test object based on a plurality of combined light beams that are obtained by combining a plurality of return light beams from the test object irradiated with a plurality of measurement light beams and a plurality of reference light beams respectively corresponding to the plurality of measurement light beams, the apparatus including: an instruction unit configured to give instructions about amounts of changes in respective optical path length differences between the plurality of reference beams and the plurality of return light beams; and a change unit configured to change the optical path length differences based on the amounts of changes instructed by the instruction unit.

### TECHNICAL FIELD

The present invention relates to an imaging apparatus and an imaging method, in particular, to an imaging apparatus and an imaging method of an optical coherence tomographic image of a test object using a plurality of measurement light beams.

### BACKGROUND ART

In recent years, in medical fields, more specifically in ophthalmic field, imaging apparatuses (hereinafter, also referred to as OCT apparatus) have been used, the apparatuses each picking up tomographic images (hereinafter, also referred to as optical coherence tomographic image) of a test object using optical coherence tomography (OCT) based on interference of low coherence light. The OCT apparatuses utilize light properties, and thereby can obtain tomographic images of high resolution with an order of light wavelength which is micrometer.

Generally, the point where the difference between an optical path length of a measurement light beam and that of a reference light beam is zero is called a coherence gate. It is essential to locate a coherence gate at a proper position on a test object's eye to obtain a tomographic image of a high signal to noise (SN) ratio and to display the tomographic image at a proper position on a monitor. Japanese Patent Application Laid-Open No. 2009-160190 discusses an OCT apparatus in which a position of a coherence gate can be specified by moving a cursor displayed on a monitor to facilitate the specification of coherence gate position by user.

While a test object's eye such as fundus is measured, the test object's movements, eye blinks, or random slight motions (i.e., involuntary eye movement during visualfixation) are inevitable. These factors deform a tomographic image of the test object's eye obtained by an OCT apparatus.

Japanese Translation of PCT International Application Publication No. 2008-508068 discusses an OCT that emits a plurality of measurement light beams to a pupil (anterior eye part) to quickly obtain an image of the three dimensional structure of the pupil. In the OCT, a radiation area per beam can be reduced, resulting in quick pickup of a three dimensional structure image.

In such imaging apparatuses that pick up optical coherence tomographic images of a test object using a plurality of measurement light beams, it is useful that each of measurement light beams (or each of optical coherence tomographic images) can be separately controlled from the viewpoint of convenience of the users. The above recited patents do not refer to improvement of convenience of the users or control of each of measurement light beams.

### CITATION LIST

### Patent Literature

PTL 1: Japanese Patent Application Laid-Open No. 2009-160190
PTL 2: Japanese Translation of PCT International Application Publication No. 2008-508068

### SUMMARY OF INVENTION

An imaging apparatus according to the present invention is able to specify a position of a coherence gate corresponding to a plurality of measurement light beams and to display each of optical coherence tomographic images of a test object using a plurality of measurement light beams, on a display unit. Thus the imaging apparatus, improves the controllability of each of measurement light beams (or each of optical coherence tomographic images) so that the imaging apparatus can be convenient for the users.

According to an aspect of the present invention, an imaging apparatus is provided, the apparatus capturing an optical coherence tomographic image of a test object based on a plurality of combined light beams that are obtained by combining a plurality of return light beams from the test object irradiated with a plurality of measurement light beams and a plurality of reference light beams respectively corresponding to the plurality of measurement light beams, and includes an instruction unit configured to give instructions about amounts of changes in respective optical path length differences between the plurality of reference light beams and the plurality of return light beams; and a change unit configured to change the optical path length differences based on the amounts of changes instructed by the instruction unit.

Further features and aspects of the present invention will become apparent from the following detailed description of exemplary embodiments with reference to the attached drawings.

### BRIEF DESCRIPTION OF DRAWINGS

The accompanying drawings, which are incorporated in and constitute a part of the specification, illustrate exemplary embodiments, features, and aspects of the invention and, together with the description, serve to explain the principles of the invention.

FIGS. 1A and 1B are block diagrams illustrating an imaging apparatus according to a first exemplary embodiment.

FIG. 2 illustrates an entire structure of the imaging apparatus according to the first exemplary embodiment.

FIGS. 3A to 3D each illustrate an image pickup area of a test object's eye captured by the imaging apparatus according to the first exemplary embodiment.

FIG. 4 is a flowchart illustrating an imaging method according to a second exemplary embodiment.

FIG. 5 illustrates display control in the imaging apparatus according to the first exemplary embodiment.

FIG. 6 illustrates display control in the imaging apparatus according to the first exemplary embodiment.

FIG. 7 illustrates a setting of position of a coherence gate according to a third exemplary embodiment.

### DESCRIPTION OF EMBODIMENTS

Various exemplary embodiments, features, and aspects of the invention will be described in detail below with reference to the drawings.

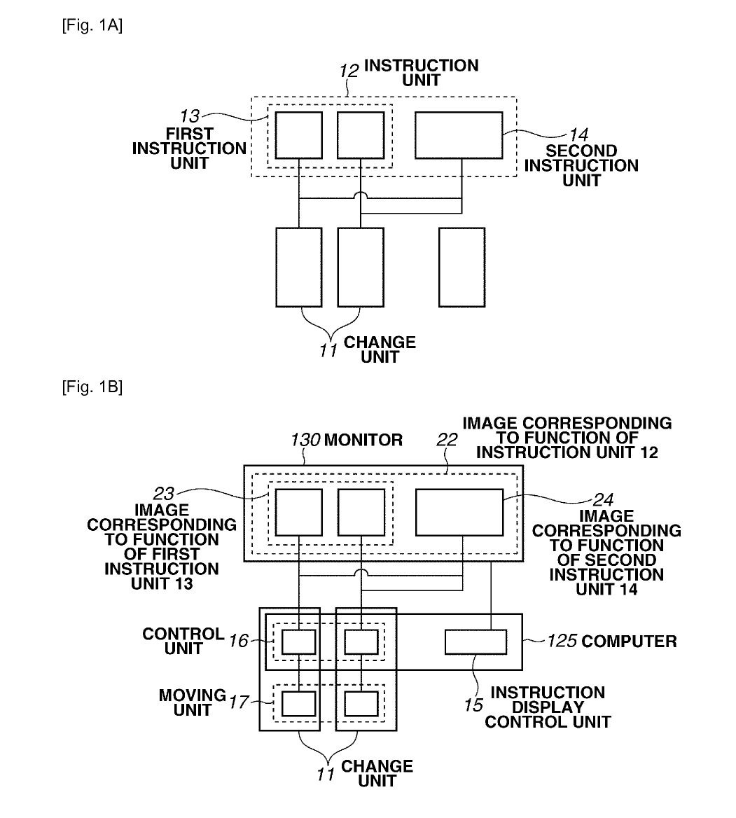

An imaging apparatus according to a first exemplary embodiment is described with reference to FIGS. 1A and 1B. FIGS. 1A and 1B are block diagrams illustrating an imaging apparatus according to the present exemplary embodiment.

An imaging apparatus according to the present exemplary embodiment captures optical coherence tomographic images of a test object using a plurality of measurement light beams. In other words, the imaging apparatus captures optical coherence tomographic images of a test object based on a plurality of combined light beams obtained by combining a plurality of return light beams from a test object that is irradiated with a plurality of measurement light beams and a plurality of reference light beams respectively corresponding to the plurality of measurement light beams. From another view point, the imaging apparatus (hereinafter, also referred to as OCT apparatus) captures tomographic images of a test object (hereinafter, also referred to as optical coherence tomographic image) by optical coherence tomography (OCT) using interference of a plurality of low coherence light beams.

An imaging apparatus according to the present exemplary embodiment includes an instruction unit 12 configured to give instructions about amounts of changes in respective optical path length differences between a plurality of reference light beams and a plurality of return light beams. In other words, the instruction unit 12 give instructions about coherence gate positions. The coherence gate position refers to a position where the above optical path length difference is zero.

The imaging apparatus according to the present exemplary embodiment further includes a change unit 11 configured to change optical path length differences based on the amounts of changes instructed by the instruction unit 12. The change unit 11 can change the optical path lengths of the reference light beams individually. The change unit 11 can include moving units 17 (e.g., movable stages 117) configured to move reference mirrors included therein respectively in an optical axis direction thereof, the mirrors being disposed in the optical paths of the plurality of reference light beams respectively. The change unit 11 may further include a control unit 16 configured to control the moving units 17. In this case, the control unit 16 may be incorporated in a computer 125.

The above structure facilitates to give instructions about an amount of change in an optical path length difference corresponding to each of the measurement light beams (or a coherence gate position). Consequently, each of measurement light beams (or each of optical coherence tomographic images) can be easily controlled, enhancing the convenience for the users.

The instruction unit 12 can include a first instruction unit 13 configured to give instructions about the amounts of changes individually, and a second instruction unit 14 configured to give instructions about the amounts of changes in association with one another. This configuration enables to specify different positions for different coherence gates individually and also to specify the positions together at one time, further enhancing the convenience for the users.

When the imaging apparatus according to the present exemplary embodiment is used to capture tomographic images of a test object's eye fundus, the imaging apparatus can further include an irradiation unit configured to irradiate the test object's eye with a plurality of measurement light beams such that the beams intersect with one another at the anterior eye part of the test object's eye (such that the anterior eye part is irradiated with the intersected beams). The irradiation unit enables irradiation of a wide area (wide angle of view) on the test object's eye fundus with a plurality of measurement light beams. In the present exemplary embodiment with the irradiation unit, the instruction unit 12 can further include a unit configured to give instructions about an amount of the change based on the features (e.g., shape and aberration) of a test object's eye. This is because the optical path lengths of a plurality of measurement light beams may be different depending on the features of a test object's eye. In such structure, coherence gate positions respectively corresponding to a plurality of measurement light beams can be individually changed, further enhancing the convenience for the users. The irradiation unit can have a scan unit (e.g., XY scanner 119) configured to scan a plurality of measurement light beams, and a light concentrating position change unit (e.g., lens 120-2) configured to change a light concentrating position in the depth direction of a fundus.

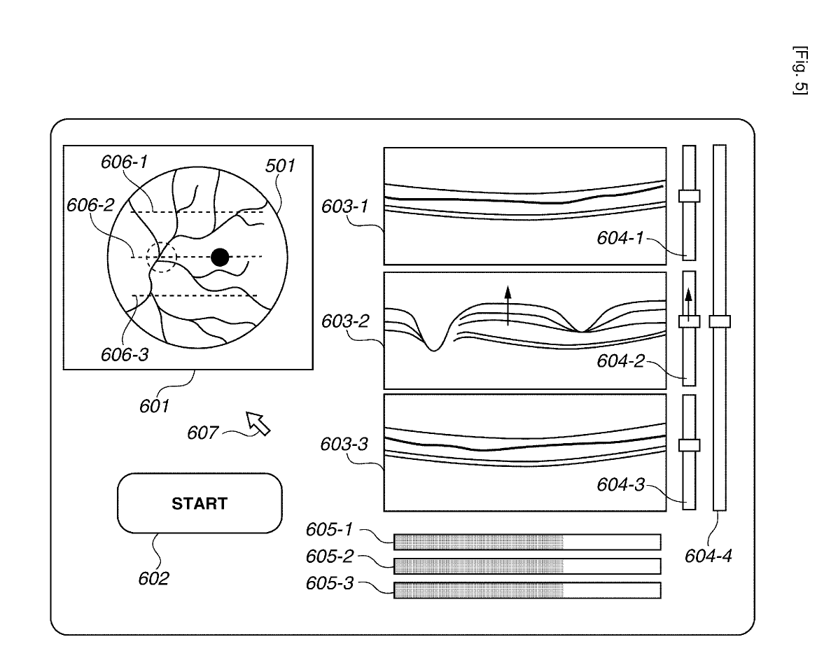

The imaging apparatus according to the present exemplary embodiment can further include, from the viewpoint of convenience of the users, an instruction display control unit 15 configured to display images 22 corresponding to functions of the instruction unit 12 on a display unit (e.g., monitor 130). The images 22 may be for example icons, sliders 604-1 to 604-4 in FIG. 5, or others in any form that can cause the functions to be executed as predetermined when clicked or dragged by a cursor on the display unit. Using the instruction display control unit 15, a user can operate a pointing device such as a mouse to give instructions about an amount of change to the change unit 11 through an image 22 corresponding to a function of the instruction unit 12. The instruction display control unit 15 can display an image 23 corresponding to a function of a first instruction unit 13 and an image 24 corresponding to a function of the second instruction unit 14 on the display unit. The display unit may be a monitor or any other apparatus that displays information based on input signals. The display unit may be in a form such that it is incorporated in the apparatus, is removable from the apparatus, or can communicate with the apparatus wirelessly or by wire.

The imaging apparatus according to the present exemplary embodiment can further include a tomographic image display control unit (not illustrated) configured to display each of tomographic images of a test object generated based on a plurality of combined light beams. The tomographic image display control unit enables the instruction display control unit 15 to display images each corresponding to the functions of the instruction units on the display unit in association with the tomographic images respectively (e.g., an image next to the associated tomographic image as illustrated in FIG. 5).

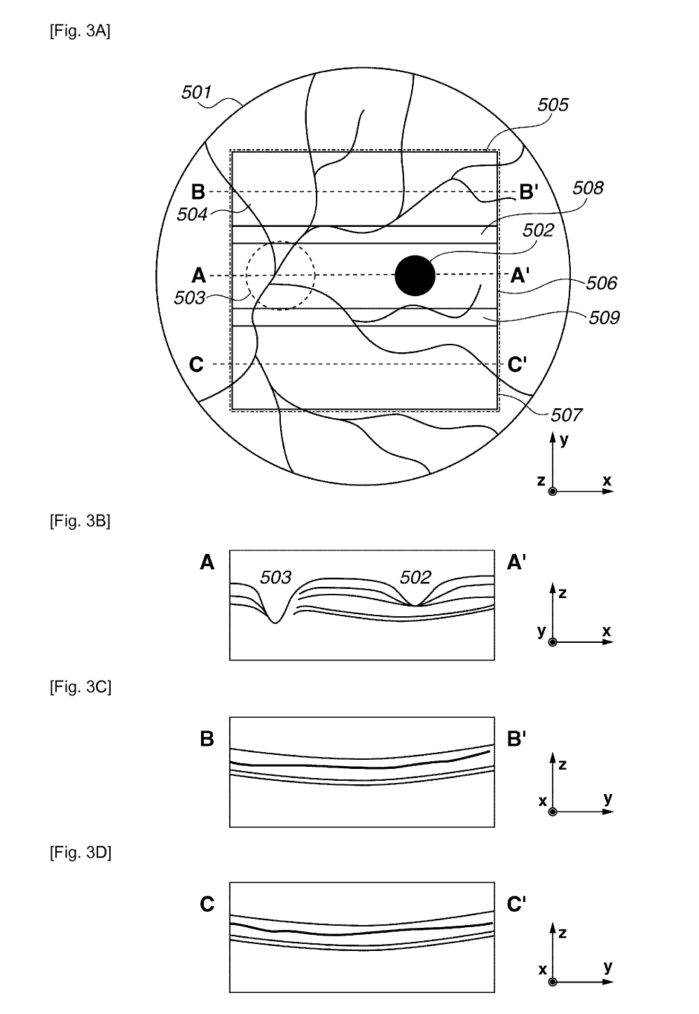

The imaging apparatus according to the present exemplary embodiment can further include an intersecting image display control unit (not illustrated) configured to display an intersecting image of a test object on the display unit, the image being captured from a direction intersecting with the direction in which the test object is irradiated with a plurality of measurement light beams. The intersecting image refers to at least one of a two dimensional image of a fundus surface (i.e., fundus image), a multiplied image of at least a part of tomographic images captured in the depth direction of a fundus, and a tomographic image (i.e., C scan image) captured in the direction approximately perpendicular to the depth direction of a fundus. When the intersecting image display control unit is provided, the present exemplary embodiment can further include a position display control unit (not illustrated) configured to display the positions of the tomographic images (e.g., scan positions 606-1 to 606-3 in FIG. 5) in association with the intersecting images respectively on the display. These units allow a user to easily recognize positional relationships between the tomographic images. The present exemplary embodiment can further include a scan range display control unit (not illustrated) configured to display scan ranges (e.g., first to third scan ranges 505 to 507 in FIGS. 3A to 3D, and scan positions 606-1 to 606-3 in FIG. 5) of the plurality of measurement light beams in association with the intersecting images respectively on the display. The scan range can be expressed as scan position, scan field, light irradiation position, and imaging area.

The present exemplary embodiment can further include an image information display control unit (not illustrated) configured to display images (e.g., bars 605-1 to 605-3 in FIG. 5) representing pieces of image information (e.g., SN ratio) respectively corresponding to the optical coherence tomographic images on the display unit.

The type of OCT applicable to the present exemplary embodiment is described. There are generally two types of OCT: Time Domain OCT (TD-OCT) and Fourier-domain OCT (FD-OCT). In the former type, a reference light beam has a controlled optical path length to change a position to capture a tomographic image, while in the latter type, data in the direction of an eye depth (the optical axis direction of an optical system) can be obtained at one time.

The Fourier-domain OCT can be divided into two types: Spectral Domain OCT (SD-OCT) and Swept Source OCT (SS-OCT). In the former type, interfered light beams are dispersed by a diffraction grating, and the dispersed beams are detected by a line sensor, while in the latter type, a wavelength tunable (i.e., capable of sweeping a wavelength) light source is used. Currently, the Spectral Domain OCT is mainly used because data in the direction of an eye depth can be obtained at higher speed than in the Time Domain OCT. An imaging apparatus according to the present exemplary embodiment may be configured to have a division unit to divide light from a light source into measurement light beams and reference light beams, and a combination unit configured to combine return light beams from a test object's eye with the reference light beams as one combined unit (as a Michelson interferometer). Alternatively, an imaging apparatus according to the present exemplary embodiment may have the division unit and the combination unit separately (as a Mach-Zehnder interferometer).

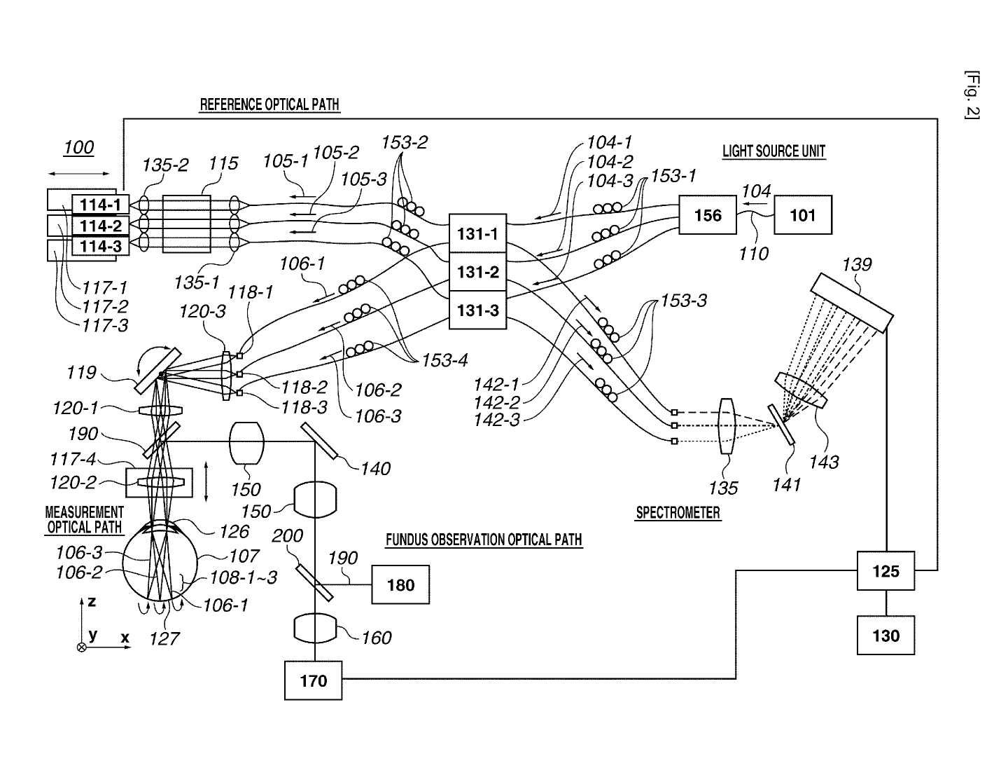

The basic structure of an imaging apparatus according to the present exemplary embodiment (hereinafter, also referred to as OCT apparatus) is described with reference to FIG. 2. An OCT apparatus 100 constitutes a Michelson interferometer as a whole, and is further a Spectral Domain OCT (hereinafter, referred to as SD-OCT). An imaging apparatus according to the present exemplary embodiment, however, is not required to be an SD-OCT, and may be of another type OCT such as a Time domain OCT. An entire structure and functions of an OCT apparatus is described below.

A light source 101 emits a light beam 104. The emitted light beam 104 travels through a single mode optical fiber 110 and enters an optical coupler 156. At the optical coupler 156, the emitted light beam 104 is divided into three emitted light beams 104-1 to 104-3 that travel through first to third optical paths respectively. The three emitted light beams 104-1 to 104-3 respectively pass through polarization controllers 153-1. At the optical couplers 131-1 to 131-3 the three emitted light beams 104-1 to 104-3 are divided into reference light beams 105-1 to 105-3 and measurement light beams 106-1 to 106-3 respectively. The three measurement light beams 106-1 to 106-3 impinge a target point to be measured such as those on a retina 127 of a test object's eye 107 to be observed, and are reflected or scattered by the points. The reflected or scattered light beams 106-1 to 106-3 return from the points as return light beams 108-1 to 108-3 respectively. The return light beams 108-1 to 108-3 are combined with the reference light beams 105-1 to 105-3 that have travelled through reference optical paths, to become combined light beams 142-1 to 142-3 respectively. The combined light beams 142-1 to 142-3 are dispersed into their constituent wavelengths by a transmission diffraction grating 141, and enter a line sensor 139. The line sensor 139 includes sensor elements each of which converts the light intensity of each wavelength into a voltage. The signals of the converted voltages are used to generate a tomography image of the test object's eye 107.

The reference optical paths for the reference light beams 105 are described. The reference light beams 105-1 to 105-3 divided at the optical couplers 131-1 to 131-3 pass through polarization controllers 153-2 respectively, and become approximately parallel to one another at the lenses 135-1, and exit the lenses. The reference light beams 105-1 to 105-3 then pass through a dispersion compensator glass 115 and lenses 135-2 to be focused on mirrors 114-1 to 114-3 respectively. The reference light beams 105-1 to 105-3 are then reflected by the mirrors 114-1 to 114-3 and travel toward the optical couplers 131-1 to 131-3 respectively again. The reference light beams 105-1 to 105-3 pass through the optical couplers 131-1 to 131-3 to the line sensor 139. The dispersion compensator glass 115 compensates the reference light beam 105 for the dispersion that is caused when the measurement light beams 106 travel the test object's eye 107 and the scanning optical system back and forth.

Motorized stages 117-1 to 117-3 moves in the directions illustrated by the arrows in FIG. 2, so that the optical path lengths of the reference light beams 105 can be changed. The motorized stages 117-1 to 117-3 are controlled by the computer 125, and change the optical path lengths of the reference light beams 105-1 to 3 separately or together at one time.

The measurement light beam paths of the measurement light beams 106 are described. The measurement light beams 106 generated at the optical couplers 131-1 to 131-3 respectively pass through the polarization controllers 153-4 and become approximately parallel light at the lens 120-3, and exit the lens to enter mirrors of an XY scanner 119 composing a scanning optical system. Only one XY scanner 119 is illustrated in FIG. 2 for simplicity, but actually an X scanning mirror and a Y scanning mirror are arranged adjacent to each other to perform raster scanning on the retina 127 in the direction perpendicular to the optical axis. The lenses 120-1 and 120-3 are adjusted such that each center of the measurement light beams 106-1 to 106-3 approximately corresponds with the rotation center of the mirrors of the XY scanner 119 respectively. The lenses 120-1 and 120-2 constitute the optical system for scanning the retina 127 using the measurement light beams 106-1 to 3, and the measurement light beams 106 scan the retina 127 with a fulcrum in the vicinity of the cornea 126. The measurement light beams 106-1 to 106-3 are each arranged to form an image on an arbitrary position on the retina 127.

The motorized stage 117-4 is movable in the direction illustrated by the arrow in FIG. 2, so that the lens 120-2 attached thereto can be adjusted and controlled to an appropriate position. The positional adjustment of the lens 120-2 allows the measurement light beams 106-1 to 106-3 to be each focused on a position on an intended layer in the retina 127 of the test object's eye 107, leading to observation of the retina 127. Entering the test object's eye 107, the measurement light beams 106-1 to 106-3 are reflected or scattered by the retina 127, and becomes return light beams 108-1 to 108-3 respectively. The return light beams 108-1 to 108-3 pass the optical couplers 131-1 to 131-3 respectively and travel to the line sensor 139. The motorized stage 117-4 is controlled by the computer 125. The above structure enables scanning using the three measurement light beams at the same time.

The structure of a detection system is described. The return light beams 108-1 to 108-3 which are reflected or scattered by the retina 127 and the reference light beams 105-1 to 105-3 are combined with one another at the optical couplers 131-1 to 131-3. The combined light beams 142-1 to 3 each enter a spectrometer to measure spectra of the beams 142-1 to 142-3. The spectra are processed by the computer 125 to be reconstructed, resulting in a tomographic image of the retina 127.

The reconstruction process may follow a typical generation process for OCT images, and a tomographic image can be obtained through fixed noise reduction, conversion of wavelength to wavenumber, and Fourier transform. FIGS. 3A to 3D illustrate an area on a fundus retina and tomographic images of the area captured by the OCT apparatus.

In FIG. 3A, the fundus 501 includes a macula 502, an optic papilla 503, and blood vessels 504. Three measurement light beams scan first to third scan ranges 505 to 507 respectively. The ranges overlap with one another by about 20% as illustrated in an overlapped region 508 between the first and second scan ranges and an overlapped region 509 between the second and third scan ranges. The coordinate axes are set as illustrated, including a scanning referred to as Fast-Scan in the x direction, a scanning referred to as Slow-Scan in the y direction, and a scanning perpendicular to the plane of the figure from the rear side to the front side in the z direction.

In the present exemplary embodiment, one measurement light beam scans 512 lines in the x direction and 200 lines in the y direction for example. In the y direction, however, three measurement light beams scan 512 lines except the overlapped regions, and eventually the rectangular area surrounded by the dashed line in FIG. 3A is the image pickup area on the fundus, and tomographic images of the area are obtained.

Meanwhile, a near infrared ray source 180 emits a near infrared light beam 190. The near infrared light beam 190 travels along a half mirror 200, an illumination optical system 150, and a dichroic mirror 190 disposed in the measurement light beam path, and illuminates the fundus 127. Reflected by the fundus 127, the infrared light beam 190 again travels the same optical path, and passes along a half mirror 200 and an image forming optical system 160 and forms an image on a two-dimensional area sensor 170. The resulting two dimensional image of the fundus is input to the computer 125. The two dimensional image is used to observe the image pickup area of fundus captured by the OCT apparatus.

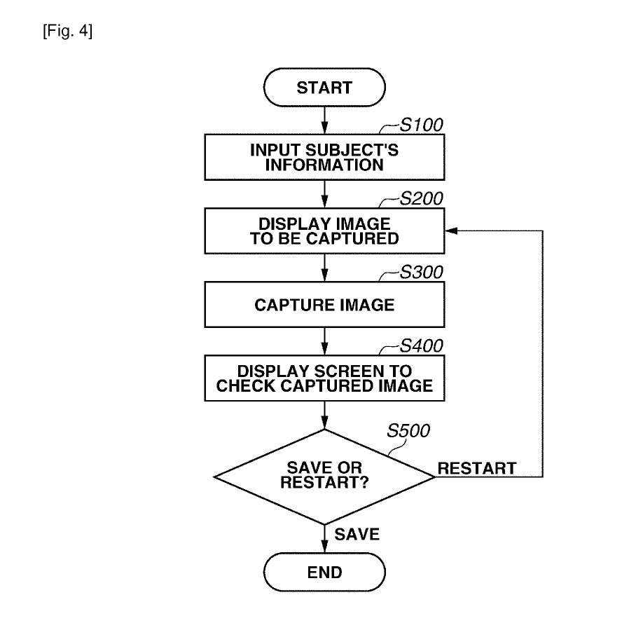

Next, alignment of measurement light beams and setting of a coherence gate position that are essential in the above described image capturing in a second exemplary embodiment are described with reference to FIG. 4. FIG. 4 is a flowchart illustrating an imaging method according to the present exemplary embodiment. The following flow is performed by an operator of an OCT apparatus through a user interface on the monitor 130 displayed by a control program (not illustrated) (hereinafter, simply referred to as control program) stored in the computer 125.

In step S100, an operator inputs information to the computer 125, the information including a patient name and a patient ID that specify a subject. Once input, the information is stored in a storage device such as a hard disk in the computer 125. Receiving the information, prior to image capturing, a control program displays a screen through which the operator determines setting of an image pickup area.

In step S200, the control program displays a user interface on the monitor 130 to set an image pickup area. FIG. 5 illustrates a user interface displayed on the monitor 130. In FIG. 5, a window 601 displays a two dimensional image of a fundus captured by the area sensor 170, and different windows 603-1 to 603-3 each continuously display a tomographic image captured using the above described three measurement light beams respectively.

The lines representing scan positions 606-1 to 606-3 of the three measurement light beams are displayed superimposing on the two dimensional image of the fundus 501 in the window 601. The operator of the OCT apparatus can specify a measurement position on the fundus 501 by moving a cursor 607 on the scan positions 606-1 to 606-3 with a pointing device such as a mouse connected to the computer 125. The specification is executed by the computer 125 through control of the rotation range of the XY scanner 119.

The operator of the OCT apparatus then adjusts coherence gate positions by manipulating the sliders 604-1 to 604-4. More specifically the motorized stages 117-1 to 117-3 can be moved by manipulation of the sliders 604-1 to 604-3, and thereby coherence gate positions of each measurement light beam can be adjusted through the manipulation by the operator. For example, the coherence gate position of the center measurement light beam displayed in the window 603-2 is adjusted through operation of the slider 604-2. As the slider 604-2 is shifted in the direction indicated by the arrow thereon in FIG. 5, the tomographic image displayed in the window 603-2 is also shifted in an upward direction.

The slider 604-4 controls the other three sliders, and manipulation of the slider 604-4 is linked to movements of the other three sliders. Accordingly, manipulation of the slider 604-4 causes all of the motorized stages 117-1 to 3 to move, so that all of the coherence gate positions of the measurement light beams are simultaneously adjusted.

Meanwhile, the windows 603-1 to 603-3 each continuously display a tomographic image obtained using the measurement light beams respectively. In each of progress bars 605-1 to 605-3, a bar is displayed, the bar having a length in proportion to an SN ratio calculated based on the corresponding tomographic image: the longer a bar extends in the right direction in FIG. 5, the higher an SN ratio. The length of each bar is updated each time a tomographic image is captured and displayed, so that the operator can adjust coherence gate positions based on the positions of the tomographic images in the window 603-1 to 603-3 and the bar lengths indicating image quality.

In step S300, after adjustment of coherence gate positions, the operator presses a start button 602 to start to capture a tomographic image. In step S400, when the capturing is completed, a resulting tomographic image is displayed on the screen for check.

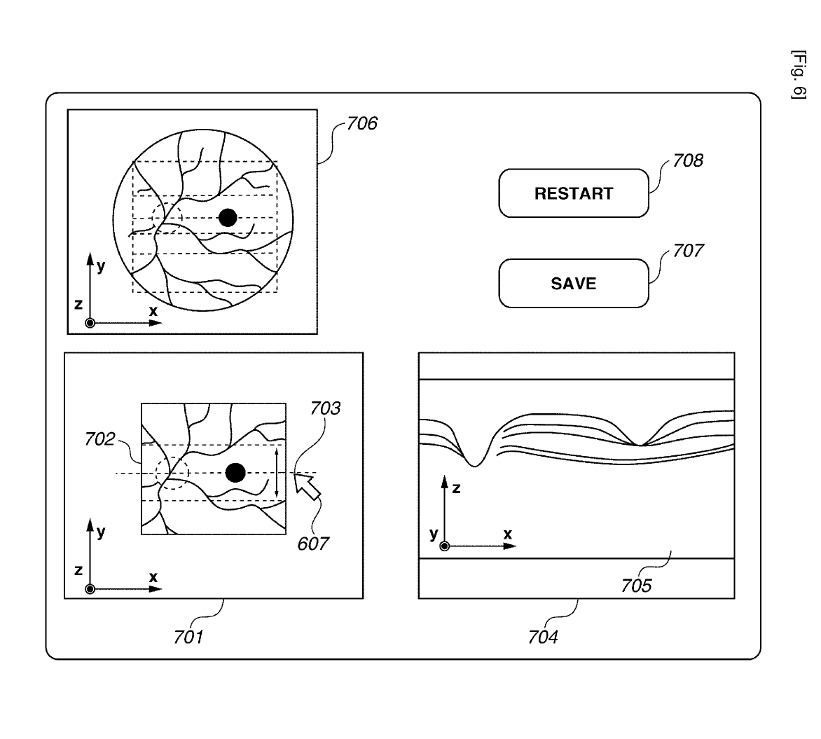

FIG. 6 illustrates a user interface on the monitor 130 after image capturing, and three windows 701, 704, and 706 each display a captured image.

The window 701 displays an integrated image 702 as a result of integrating pixel values of a tomographic image in the z direction (depth direction) of the test object's eye. Pixel values of a tomographic image of retina captured by an OCT is approximately proportional to a reflectance at the interface of each layer (difference in refractive index between layers) in the retina. Therefore the integrated image of the pixel values in the z direction of a target fundus is extremely similar to the two dimensional image of the fundus. Accordingly, display of the captured integrated image allows an operator to check for troubles of the measurement light beams due to shading (vignetting) for example in the anterior eye part (e.g., cornea).

In the present exemplary embodiment, three measurement light beams are used, and thereby the overlapped regions 508 and 509 in FIG. 3A are not displayed in a projected image. The overlapped regions may be removed from an image by any method. For example, the center area may be left as is and the upper and lower overlapped areas of each area may be removed to form three integrated images, and the integrated images can be connected to form one integrated image. In formation of an integrated image, as illustrated in an integrated image 702 in FIG. 6, the borders between areas can be indicated by dashed lines for example. This allows a user to recognize which area of a fundus is captured with each of the three measurement light beams.

A window 704 displays a tomographic image 705 for the point specified through a cursor 703 on the integrated image 702. The position of the displayed point can be changed by operating the cursor 703 using a pointing device such as a mouse. This allows a user to observe the overall fundus using integrated image 702 and also to check individual tomographic images.

A window 706 displays a fundus image detected by an area sensor 170 of a fundus observing optical system. On the fundus image also, the borders between the areas captured using the measurement light beams are displayed by a rectangular dashed line, for example, to be recognized by a user. The rectangular areas can have different colors, making the areas more recognizable.

After completing check of a tomographic image in step S500, an operator presses a save utton 707 to store the image in a storage device of the computer 125. If capturing of a new tomographic image is necessary, an operator presses a restart button 708 to return the process to step S200 to capture a new tomographic image.

As described above, an OCT apparatus using a plurality of measurement light beams includes a user interface provided with: units that correspond to the measurement light beams respectively and are configured to adjust coherence gates; and a unit configured to totally adjust the measurement light beams at one time. Thus, convenience of the apparatus for the user can be enhanced, and capturing of images of high SN ratio can be achieved.

In the present exemplary embodiment, sliders are used to adjust coherence gate positions, but the method is not limited to that and other approaches may be used. For example, scroll bars may be used. Alternatively, another operation panel may be provided to the body of an OCT apparatus, so that a plurality of dials is arranged on the panel to adjust coherence gates.

In the present exemplary embodiment, on the check screen, after image capturing, a captured tomographic image is displayed together with an integrated image to be generated and a fundus image, thereby facilitating the checking whether the imaging is normal.

AS a third exemplary embodiment, a method of setting a coherence gate position based on a test object's eye is described. In the above exemplary embodiments, coherence gate positions are set by operating corresponding sliders, but in the case where images of the test object's eye have been already captured, the images can be used to automatically set the initial position of each of the sliders. This can save considerable time in adjusting coherence gates.

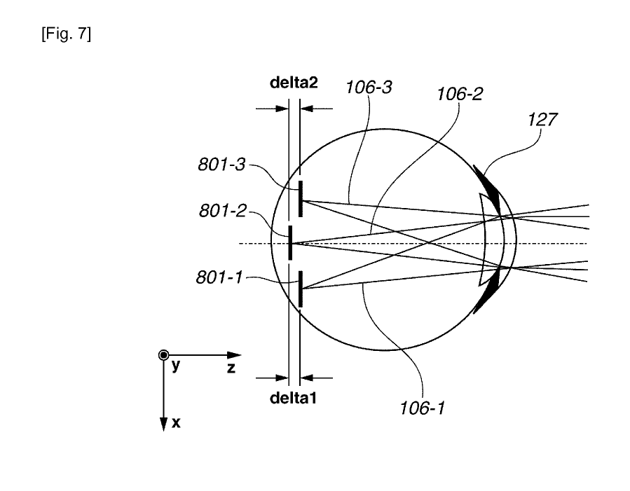

FIG. 7 illustrates positional relationship between the measurement light beams 106-1 to 106-3 and the test object's eye 127, and illustrates coherence gate positions 801-1 to 801-3 corresponding to the measurement light beams respectively. The coherence gate positions can be, as described in the first exemplary embodiment, changed by operating the slider 604-1 to 604-3 on the user interface and moving the motorized stages 117-1 to 117-3. In the present exemplary embodiment, the adjusted coherence gate positions relative to a test object's eye are stored in an OCT apparatus in association with the test object's eye.

More specifically, the position of the coherence gate 801-2 for the center measurement light beam 106-2 is stored as positional information of the motorized stage 117-2, and also a difference delta 1 between the coherence gates 801-2 and 801-1, and a difference delta 2 between the coherence gates 801-2 and 801-3 are stored in a storage device of the computer 125 connected to the OCT apparatus, together with identification information of the test object's eye. This storage is executed by a control processing unit (CPU) of the computer 125 based on a program that controls the OCT apparatus.

When images of the same test object's eye are captured next time, the CPU reads the identification information of the test object's eye, the position of the coherence gate 802-2, and delta 1, delta 2 from the memory, so that the pieces of the information are represented by the initial positions of the slider 604-1 to 604-3 on a user interface, and the motorized stages 117-1 to 117-3 are driven to automatically set the coherence gate positions.

As a result, the relative positional relationship between the coherence gates returns to the state identical to that at the previous image capturing, and thereby, basically, only adjustment of the position of the test object's eye relative to the OCT apparatus is required: an operator is only required to adjust the slider 604-4, which simplifies the operation for image capturing.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2010-082814 filed Mar. 31, 2010, which is hereby incorporated by reference herein in its entirety.

1. An imaging apparatus capturing optical coherence tomographic images of a test object based on a plurality of combined light beams that are obtained by combining a plurality of return light beams from the test object irradiated with a plurality of measurement light beams and a plurality of reference light beams respectively corresponding to the plurality of measurement light beams, the apparatus comprising:
an instruction unit configured to give instructions about amounts of changes in respective optical path length differences between the plurality of reference light beams and the plurality of return light beams; and
a change unit configured to change the optical path length differences based on the amounts of changes instructed by the instruction unit.

2. The imaging apparatus according to claim 1, wherein the instruction unit includes a first instruction unit configured to give instructions about the amounts of changes individually, and a second instruction unit configured to give instructions about the amounts of changes in association with one another.

3. The imaging apparatus according to claim 2, wherein the second instruction unit gives instructions about the amounts of changes at one time.

4. The imaging apparatus according to claim 1, wherein the change unit changes optical path lengths of the plurality of reference light beams individually.

5. The imaging apparatus according to claim 1, wherein the change unit includes a moving unit configured to move reference mirrors in the direction of optical axes of the mirrors respectively, the mirrors being provided in optical paths of the plurality of reference light beams respectively.

6. The imaging apparatus according to claim 1, wherein the test object is a test object's eye,
the imaging apparatus further comprising an irradiation unit configured to irradiate the test object's eye with the plurality of measurement light beams such that the plurality of measurement light beams intersect with one another at the anterior eye part of the test object's eye, and
wherein the instruction unit includes a unit configured to give instructions about the amounts of changes based on features of the test object's eye.

7. The imaging apparatus according to claim 1, further comprising an instruction display control unit configured to display an image corresponding to a function of the instruction unit on a display unit.

8. The imaging apparatus according to claim 7, further comprising a tomographic image display control unit configured to display respective optical coherence tomographic images of the test object based on the plurality of combined light beams on the display unit,
wherein the instruction display control unit displays images respectively corresponding to functions of the instruction unit in association with the optical coherence tomographic images respectively on the display unit.

9. The imaging apparatus according to claim 8, further comprising: an intersecting image display control unit configured to display an intersecting image of the test object on the display unit, the intersecting image being captured in a direction intersecting with the direction in which the test object is irradiated with the plurality of measurement light beams; and
a position display control unit configured to display positions of the respective optical coherence tomographic images in association with the intersecting image on the display unit.

10. The imaging apparatus according to claim 8, further comprising an image information display control unit configured to display images respectively representing pieces of information corresponding to the optical coherence tomographic images on the display unit.

11. The imaging apparatus according to claim 7, further comprising: an intersecting image display control unit configured to display an intersecting image of the test object captured in a direction intersecting with the direction in which the test object is irradiated with the plurality of measurement light beams, on the display unit; and
a scanning area display control unit configured to display scanning areas of the plurality of measurement light beams in association with the intersecting image on the display unit.

12. An imaging apparatus for capturing optical coherence tomographic images of a test object based on a plurality of combined light beams that are obtained by combining a plurality of return light beams from the test object irradiated with a plurality of measurement light beams and a plurality of reference light beams respectively corresponding to the plurality of measurement light beams, the apparatus comprising:
a first instruction unit configured to give instructions about amounts of changes in respective optical path length differences between the plurality of reference light beams and the plurality of return light beams;
a second instruction unit configured to give instructions about the amounts of changes in association with one another; and
an instruction display control unit configured to display an image corresponding to a function of the first instruction unit and an image corresponding to a function of the second instruction unit on a display unit.

13. An imaging apparatus for capturing optical coherence tomographic images of a test object based on a plurality of combined light beams that are obtained by combining a plurality of return light beams from the test object irradiated with a plurality of measurement light beams and a plurality of reference light beams respectively corresponding to the plurality of measurement light beams, the apparatus comprising:
a tomographic image display control unit configured to display optical coherence tomographic images of the test object based on the plurality of combined light beams on a display unit; and
an image information display control unit configured to display images respectively representing pieces of information corresponding to the optical coherence tomographic images on the display unit.

14. An imaging method of capturing optical coherence tomographic images of a test object using the imaging apparatus according to claim 7, comprising:
inputting a signal to be displayed on a display unit; and
displaying the information based on the input signal on the display unit.

15. An imaging method of capturing optical coherence tomographic images of a test object based on a plurality of combined light beams that are obtained by combining a plurality of return light beams from the test object irradiated with a plurality of measurement light beams and a plurality of reference light beams respectively corresponding to the plurality of measurement light beams, comprising:
giving instructions about amounts of changes in respective optical path length differences between the plurality of reference light beams and the plurality of return light beams; and
changing the optical path length differences based on the instructed amounts of changes.

16. The imaging method according to claim 15, wherein the instruction includes a first instruction that gives instructions about the amounts of changes individually, and a second instruction that gives instructions about the amounts of changes in association with one another.

17. The imaging method according to claim 15, wherein, in the changing of the optical path length differences, optical path lengths of the plurality of reference light beams are individually changed.

18. A program executing the imaging method according to claim 14 on a computer.
